(19) United States
(12) Patent Application Publication
(10) Pub. No.: US 2019/0060179 A1
(43) Pub. Date: Feb. 28, 2019

(54) BUCCAL CAVITY FEEDING SYSTEM

(71) Applicant: William J. Hermann, JR., Sealy, TX (US)

(72) Inventor: William J. Hermann, JR., Sealy, TX (US)

(21) Appl. No.: 15/693,143

(22) Filed: Aug. 31, 2017

(51) Int. Cl. A61J 15/00

(52) U.S. Cl. CPC A61J 15/0026; A61M 16/0488; A61J 15/0011

(57) ABSTRACT

An apparatus and method for oral feeding that includes an orally insertable tube for release of fluid into the mouth, i.e. within the buccal cavity, between the cheek and teeth. The apparatus includes a rigid or semi-rigid flat or outwardly curved side for positioning against the teeth and a spongy side for positioning against the cheek. The tube may be connected to a reservoir disposed outside of the mouth. Fluid within the tube may be distributed into the mouth by the feeding tube through the spongy side to provide a distributed flow of fluid into the mouth and to avoid overwhelming the patient's ability to swallow or having a gush of fluid reach the back of the throat of the patient. Fluid flow into the tube may be manually controlled by valve disposed downstream of the reservoir.

### BACKGROUND OF THE DISCLOSURE

### 1. Field of the Disclosure

This disclosure relates to the field of medical devices and, more specifically, oral feeding devices.

### 2. Description of the Related Art

Feeding systems are used to provide supporting fluids, such as water, liquefied food, and nutrient solutions to patients that are unable to feed themselves. The difficulty in self feeding may arise from the patient begin injured, partially paralyzed, or suffering from other physical or mental conditions.

In some cases, the patient may be able to swallow and desire to receive fluids orally instead of intravenously. Oral consumption of solid food and liquids can maintain the functioning of the digestive system during periods of hospitalization or convalescence, which is not performed when all nutrition is received intravenously.

A shortcoming of prior art intraoral feeding systems is the tendency of these feeding systems to provide a gush or burst of fluid to the back of the mouth that can overwhelm the patient's capacity to orally receive fluid, such as by swallowing. Being overwhelmed by the gush of fluid can cause the patient stress and strain as they cough and gag to clear the throat and windpipe of excess fluid. Subsequently, the excess fluid being cleared may result in drooling or expiration from the mouth.

Another shortcoming of the prior art is not providing a control mechanism for patients or caretakers to manually pump a controllable amount of fluid into the patient's mouth. Many systems involve automated pumps that continuously dispense fluid into the mouth of the patient.

For these reasons there is a need to develop a buccal cavity feeding device that provides manual control to a patient or caregiver and does not overwhelm the patient's throat with fluid.

### BRIEF SUMMARY OF THE DISCLOSURE

In aspects, the present disclosure is related to methods and apparatuses for providing fluids to medical patients, specifically for intraoral feeding of medical patients.

One embodiment according to the present disclosure includes an oral fluid transfer system, comprising: a buccal cavity insert, comprising: an open cell foam body dimensioned for placement in a human mouth between an inside of a cheek and an outside of gums; a tube inserted into the open cell foam body, wherein a portion of the tube inserted into the open cell foam body has at least one opening; and wherein the open cell foam body comprises a medial side and a lateral side, the lateral side being shaped to conform to the interior of a cheek; and a first fluid reservoir in fluid communication with the tube. The system may include a second fluid reservoir in fluid communication with the first fluid reservoir. The first fluid reservoir may be a hand bulb pump. The hand bulb pump is sized to hold a predetermined amount of fluid. The open cell foam body may have a lateral side opposite the medial side, and the lateral side may be shaped to conform to the shape of the gums. The lateral side may be curved inward. The medial side may be flat or curved outward. There may be at least one opening at an end of the portion of the tube inserted into the open cell foam body, at least one opening in the side of the tube, or both. The system may include at least one clip coupled to the tube and configured to engage a human cheek.

Another embodiment according to the present disclosure is an oral feeding apparatus, comprising: a buccal cavity insert comprising: an open cell foam body dimensioned for placement in a human mouth between an inside of a cheek and an outside of gums; a tube inserted into the open cell foam body, wherein a portion of the tube inserted into the open cell foam body has at least one opening; and wherein the open cell foam body comprises a medial side and a lateral side, the lateral side being shaped to conform to the interior of a cheek. The open cell foam body has a lateral side opposite the medial side, and the lateral side is shaped to conform to the shape of the gums. The lateral side may be curved inward. The medial side may be flat or curved outward. There may be at least one opening at an end of the portion of the tube inserted into the open cell foam body, at least one opening in the side of the portion of the tube inserted into the open cell foam body, or both. The system may include at least one clip coupled to the tube and configured to engage a human cheek. The system may include a backing layer disposed against the lateral side.

Examples of the more important features of the disclosure have been summarized rather broadly in order that the detailed description thereof that follows may be better understood and in order that the contributions they represent to the art may be appreciated. There are, of course, additional features of the disclosure that will be described hereinafter and which will form the subject of the claims appended hereto.

### BRIEF DESCRIPTION OF THE DRAWINGS

A better understanding of the present disclosure can be obtained with the following detailed descriptions of the various disclosed embodiments in the drawings, which are given by way of illustration only, and thus are not limiting the present disclosure, and wherein:

FIG. 1 shows a diagram of an oral fluid transfer system according to one embodiment of the present disclosure;

FIG. 2A shows a longitudinal diagram of a buccal cavity insert in accordance with FIG. 1;

FIG. 2B shows a transverse diagram of the buccal cavity insert in accordance with FIG. 1;

FIG. 2C shows a longitudinal diagram of an alternative buccal cavity insert in accordance with FIG. 1;

FIG. 2D shows a transverse diagram of the alternative buccal cavity insert in accordance with FIG. 1;

FIG. 3A shows a diagram of the buccal cavity insert of FIG. 2B positioned within a human buccal cavity;

FIG. 3B shows a diagram of the alternative buccal cavity insert of FIG. 2D positioned within a human buccal cavity;

FIG. 4 shows a diagram of an embodiment of the fluid reservoir in accordance with FIG. 1;

FIG. 5 shows a diagram of the buccal cavity insert of FIG. 2A positioned against a human cheek in accordance with FIG. 1; and

FIG. 6 shows a flow chart of an embodiment of a method of providing fluid to a patient using the system of FIG. 1.

While the inventions disclosed herein are susceptible to various modifications and alternative forms, only a few specific embodiments are shown by way of example in the drawings and are described in detail below. The figures and detailed descriptions of these specific embodiments are not intended to limit the breadth or scope of the inventive concepts or the appended claims in any manner. Rather, the figures and detailed written descriptions are provided to illustrate the inventive concepts to a person of ordinary skill in the art, and to enable such persons to make and use one or more of the inventive concepts.

### DETAILED DESCRIPTION OF THE DISCLOSURE

In aspects, the present disclosure is related to an apparatus for feeding patients. Specifically, the present disclosure is related to an oral feeding device used to deliver liquids or other fluid substances to a patient. The present invention is susceptible to embodiments of different forms. There are shown in the drawings, and herein will be described in detail, specific embodiments with the understanding that the present invention is to be considered an exemplification of the principles and is not intended to limit the present invention to that illustrated and described herein.

One or more illustrative embodiments incorporating the invention disclosed herein are presented below. Not all features of an actual implementation are described or shown in this application for the sake of clarity. It is understood that in the development of an actual embodiment incorporating the present invention, numerous implementation-specific decisions must be made to achieve the developer's goals, such as compliance with system-related, business-related, government-related and other constraints, which vary by implementation from time to time. While a developer's efforts might be complex and time consuming, such efforts would be, nevertheless, a routine undertaking for those of ordinary skill in the art having the benefit of this disclosure.

Accordingly, it is an object of the present disclosure to provide an oral feeding device to enable patients with varying degrees of facial paralysis to receive fluids without a threat of overwhelming the patient. This threat is mitigated, in part, by the design of the oral feeding device to disperse fluids within the patient's mouth and by providing a control device to govern the amount of fluid introduced through the oral feeding device.

While apparatuses and methods are described in terms of “comprising” various components or steps (interpreted as meaning “including, but not limited to”), the compositions and methods can also “consist essentially of” or “consist of” the various components and steps, such terminology should be interpreted as defining essentially closed-member groups.

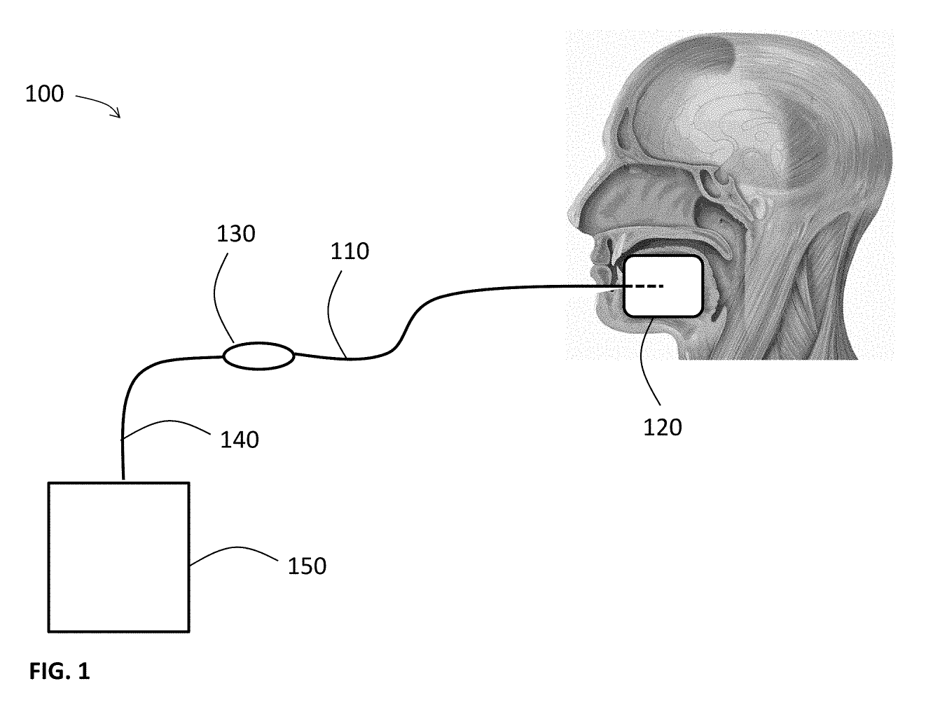

FIG. 1 shows a buccal cavity feeding apparatus 100, which includes a tube 110 and a fluid distribution pad 120. The combination of the tube 110 and the fluid distribution pad 120 may form an insert configured to fit inside the patient's buccal cavity. The fluid distribution pad 120 surrounds the tube 110, which terminates within the fluid distribution pad 120. The tube 110 may be connected to a first fluid reservoir 130. The first fluid reservoir 130 may be a squeezable bulb, that can be operated by hand or machine. The squeezable bulb may be sized based on the patient's ability or desire to receive a manageable amount of fluid with each full or partial squeeze of the bulb. The first fluid reservoir 130 may, optionally, be filled through a tube 140 that is connected to an optional second fluid reservoir 150. The first fluid reservoir 130 may include one-way valves at the entry and exit of the squeezable bulb so that the predetermined volume of liquid is pumped through the tube 110 with each squeeze of the first fluid reservoir 130. Optionally, the first fluid reservoir 130 may include a volume or flow control to aid the patient in managing the volume of fluid pumped with each squeeze of the squeezable bulb. In some embodiments, the second fluid reservoir 150 may include a pump or other fluid moving device to transport fluid from the second fluid reservoir 150 to the first fluid reservoir 130.

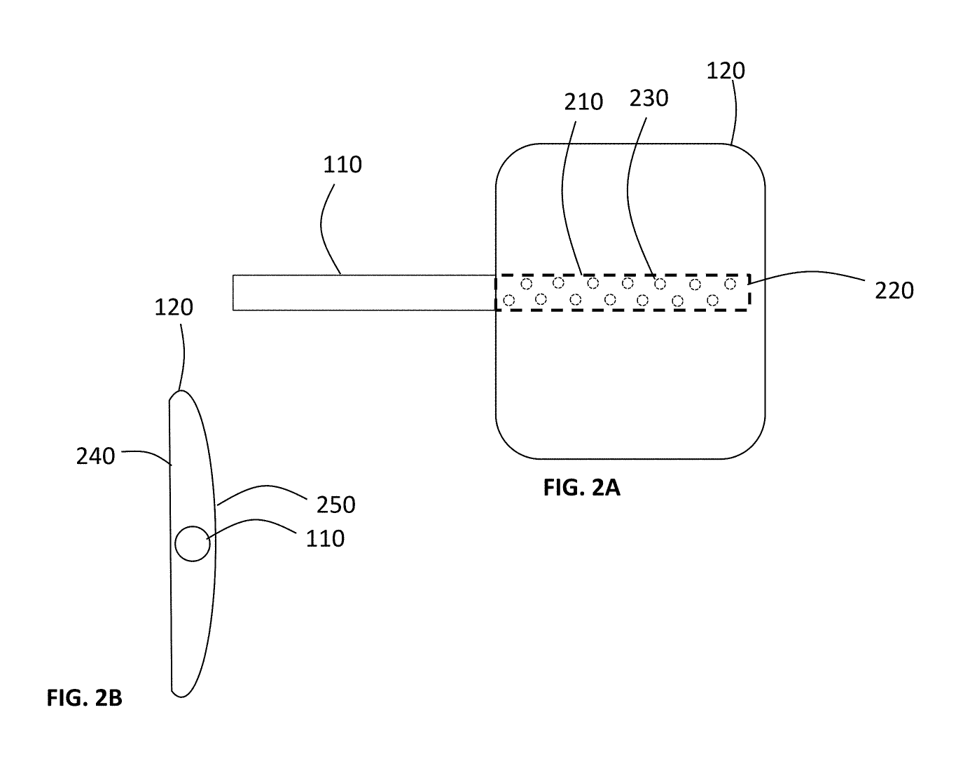

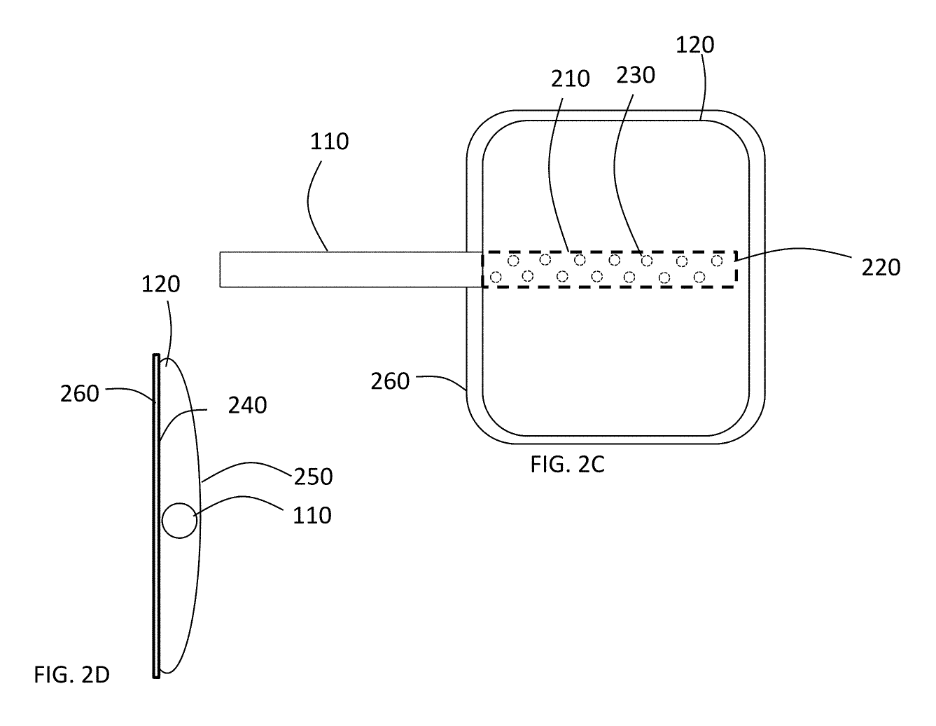

FIG. 2A shows a diagram of the tube 110 and the fluid distribution pad 120. The fluid distribution pad 120 may be made of an open cell or porous spongy material that allows fluid to flow through an indirect path. In some embodiments, the fluid distribution pad 120 may be made of a plastic polymer such as, but not limited to, polyvinyl alcohol or polyurethane. In some embodiments, the pore size of the spongy material may be selected based on desired flow characteristics to efficiently transfer fluid into a patient's buccal cavity by manual squeezing force without allowing flow in such amounts as to overwhelm the ability of the patient to swallow or otherwise manage the fluid intake. The tube 110 may have an inserted portion 210 that is embedded in the fluid distribution pad 120. The inserted portion 210 of the tube 110 may include an end opening 220 at the furthest extent of the tube 110 into the fluid distribution pad 120. The inserted portion 210 may include one or more openings 230 in the walls of the tube 110. In some embodiments, the tube inserted portion 210 may have the side openings 230 and not the end opening 220. In some embodiments, the tube inserted portion 210 may have an end opening 220 but no side openings 230. In some embodiments, the inserted portion 210 may extend substantially to the end of the fluid distribution pad 120 so that there is not a large volume of open cell foam at the end opening 220 that may accumulate fluid and make it difficult to control flow and volume.

FIG. 2B shows a transverse view of the tube 110 and the fluid distribution pad 120 of FIG. 2A. The fluid distribution pad 120 may have a first side 240 and a second side 250 (for clarity, the first and second sides are hereinafter discussed as the medial side 240 and the lateral side 250 in reference to the relationship of the fluid distribution pad 120 to the patient when the fluid distribution pad 120 is positioned within the mouth of the patient). The medial side 240 may be either substantially flat or curved outward. The lateral side 250 may be curved inward. In some embodiments, the inward curved shape of the lateral side 250 will conform to the shape of the inside of a human cheek. In some embodiments, the fluid distribution pad 120 may be uniform in composition.

FIG. 2C shows a diagram of an alternative embodiment of the fluid distribution pad 120 as shown in FIG. 2A with a backing layer 260 disposed on the medial side 240 of the fluid distribution pad 120. The backing layer 260 may be made of the same or different material as the fluid distribution pad 120; however, the backing layer 260 is structured to be less pliable than the spongy material of the fluid distribution pad 120. The backing layer 260 is rigid or resilient and helps the fluid distribution pad 120 keep its shape on the medial side 240 when pressed against the teeth or gums. The backing layer 260 may be porous or nonporous. The backing layer 260 also helps to protect the fluid distribution pad 120 and the tube 110 from the teeth 350 of the patient. Some patients, intentionally or unintentionally (such as with spastic disorders), may clench their teeth 350 and damage or interfere with the flow characteristics of the fluid distribution pad 120 and the tube 110.

FIG. 2D shows a transverse view of the alternative embodiment in FIG. 2C. The backing layer 260 may be flat or curved outward in order to conform to the shape of the medial side 240 or to give shape to the medial side 240 of the fluid distribution pad 120. The backing layer 260 is shown as extending beyond the ends of the medial side 240 of the fluid distribution pad 120; however, this is illustrative and exemplary only as other configurations are contemplated such as the backing layer 260 conforming to the dimensions of the medial side 240 or having a smaller surface area than the medial side 240.

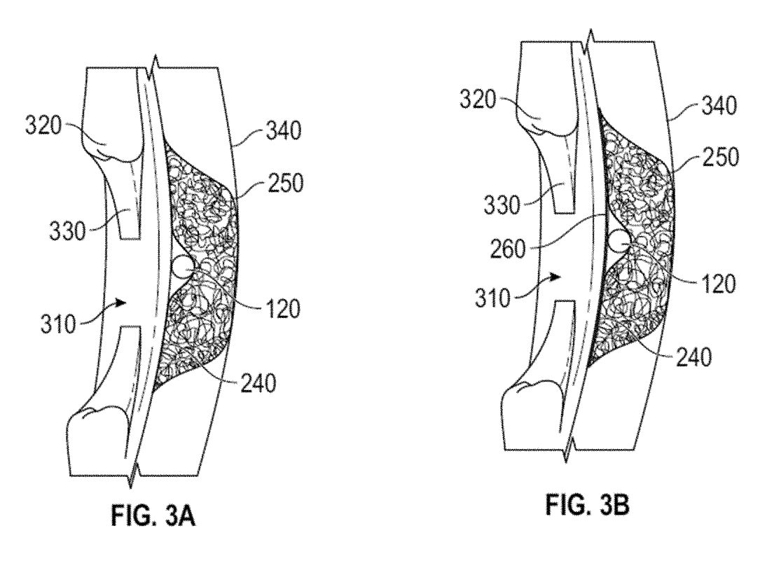

FIG. 3A shows an end view of the tube 110 and fluid distribution pad 120 of FIG. 2B positioned in a buccal cavity 310 of a patient's mouth. The medial side 240 may be positioned opposite the gums 320 and teeth 330 of the patient. The lateral side 250 may be positioned opposite the cheek 340 of the patient. In some embodiments, the lateral side 250 may conform to the shape of the cheek 340 when placed in the buccal cavity 310.

FIG. 3B shows an end view of the tube 110 and fluid distribution pad 120 of FIG. 2D positioned in the buccal cavity 310 of the patient's mouth. The backing layer 260 may be positioned opposite the gums 320 and teeth 330 of the patient. The lateral side 250 may be positioned opposite the cheek 340 of the patient. In some embodiments, the lateral side 250 may conform to the shape of the cheek 340 when placed in the buccal cavity 310. The backing layer 260 is shown as curved outward, though it may be flat in some embodiments. The backing layer 260 and the medial side 240 may be less curved than the lateral side 250.

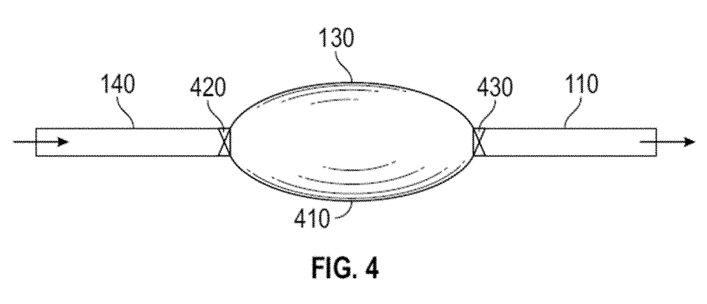

FIG. 4 shows an embodiment of the first fluid reservoir 130. The first fluid reservoir 130 may include a squeezable hand pump. The first fluid reservoir 130 may include a liquid secure housing 410 and be equipped with a one-way inlet valve 420 and a one-way outlet valve 430 so that fluid can be drawn into the liquid secure housing 410 through the one-way inlet valve 420 and pushed out of the liquid secure housing 410 through the one-way outlet valve 430 when the liquid secure housing 410 is compressed. In some embodiments, the liquid secure housing 410 is a squeezable bulb. In some embodiments where the first fluid reservoir 130 includes a manually squeezable pump, the one-way outlet valve 430 may be adjustable to control the rate flow through the one-way outlet valve 430 when the bulb is squeezed, such as by restricting the degree to which the one-way outlet valve 430 may be opened. The use of manual squeeze hand pump as the first fluid reservoir 130 is exemplary and illustrative only, as other suitable liquid reservoirs known to persons of ordinary skill in the art may be used. For example, the first fluid reservoir 130 may include a stationary tank with an automated pump that includes a control switch to activate the pump for a metered amount of liquid to be dispensed (not shown).

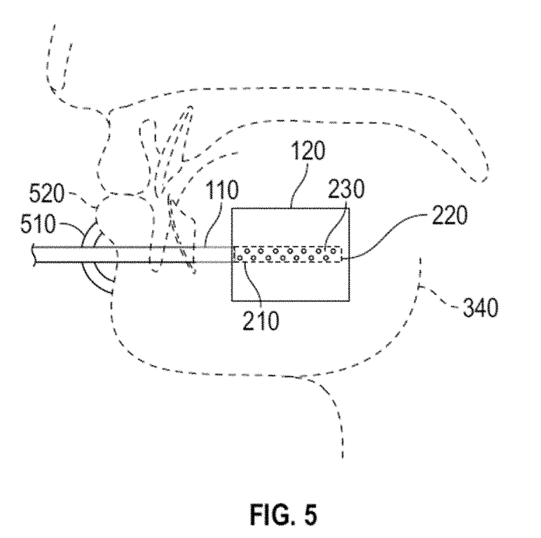

FIG. 5 shows the tube 110 and the fluid distribution pad 120 positioned within the buccal cavity and against the cheek 340. One or more adjustable clips 510 may be coupled to the tube 110 and configured to be releasably coupled to the outside or corner of the mouth 520 or to the cheek 340.

All of the apparatuses disclosed and claimed herein can be made and executed without undue experimentation in light of the present disclosure. While the methods and apparatus of this invention have been described in terms of preferred embodiments, it will be apparent to those of skill in the art that variations may be applied to the methods, processes and/or apparatus and in the steps or in the sequence of steps of the methods described herein without departing from the concept and scope of the invention. More specifically, it will be apparent that certain features which are both mechanically and functionally related can be substituted for the features described herein while the same or similar results would be achieved. All such similar substitutes and modifications apparent to those skilled in the art are deemed to be within the scope and concept of the invention.

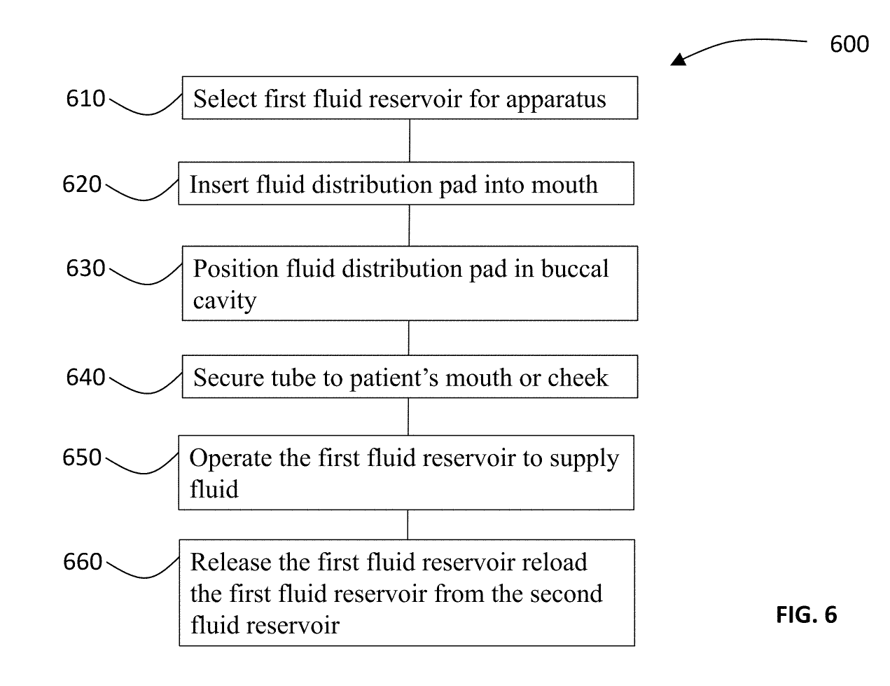

FIG. 6 shows a flow chart for a method 600 of transporting fluid into the buccal cavity of a patient according to the present disclosure. In step 610, the first fluid reservoir 130 is selected for the apparatus 100 to be used with the patient. The first fluid reservoir 130 may be selected based on volume and/or flow characteristics. The volume and/or flow characteristics required may be determined by the size of the patient, the ability of the patient to control their facial muscles and swallowing, and the person intended to operate the first fluid reservoir 130 (patient or caregiver). In step 620, the fluid distribution pad 120 (with tube 110 inserted) is inserted into the mouth of the patient. In step 630, the fluid distribution pad 120 is positioned in the buccal cavity such that the lateral side 250 is proximate to the cheek and the medial side 240 is proximal to the teeth and/or gums. In embodiments where a backing layer 260 is associated with the medial side 240, the backing layer is positioned proximal to the teeth and/or gums as well. The outward curvature of the medial side 240 and/or the backing layer 260 prevents the clenching of teeth or spastic movements of the jaw from allowing the patient to bite or otherwise damage the fluid distribution pad 120. In step 640, the tube 110 may be secured to the mouth 520 of the patient using clips or other fasteners 510 to prevent the tube 110 and fluid distribution pad 120 from being dislodged from the mouth 520 and buccal cavity 340, respectively. In step 650, the first fluid reservoir 130 maybe operated to supply fluid to the patient. The operation may include squeezing of the liquid secure housing 410 of the first fluid reservoir 130 to force fluid through the one-way outlet valve 430 and into the tube 110. In step 660, when a second fluid reservoir 150 is present, the first fluid reservoir 130 may be reloaded by releasing the liquid secure housing 410 to generate suction that draws fluid from the second fluid reservoir 150 through the tube 140 and one-way inlet valve 420. Steps 650 and 660 may be repeated as necessary to provide fluid to the patient. Step 660 may be initially performed before step 650 to load the first fluid reservoir 130 for performing step 650 the first time.

While embodiments in the present disclosure have been described in some detail, according to the preferred embodiments illustrated above, it is not meant to be limiting to modifications such as would be obvious to those skilled in the art.

The foregoing disclosure and description of the disclosure are illustrative and explanatory thereof, and various changes in the details of the illustrated apparatus and system, and the construction and the method of operation may be made without departing from the spirit of the disclosure.

What is claimed is:

1. An oral fluid transfer system, comprising:
a buccal cavity insert, comprising:
an open cell foam body dimensioned for placement in a human mouth between an inside of a cheek and an outside of gums;
a tube inserted into the open cell foam body, wherein a portion of the tube inserted into the open cell foam body has at least one opening; and
wherein the open cell foam body comprises a medial side and a lateral side, the lateral side being shaped to conform to the interior of a cheek; and
a first fluid reservoir in fluid communication with the tube.

2. The system of claim 1, further comprising:
a second fluid reservoir in fluid communication with the first fluid reservoir.

3. The system of claim 1, wherein the first fluid reservoir is a hand bulb pump.

4. The system of claim 1, wherein the hand bulb pump is sized to hold a predetermined amount of fluid.

5. The system of claim 1, wherein the lateral side is curved inward.

6. The system of claim 1, wherein the medial side is shaped to conform to the gums.

7. The system of claim 1, wherein the medial side is one of: flat and curved outward.

8. The system of claim 1, wherein the at least one opening is at an end of the portion of the tube inserted into the open cell foam body.

9. The system of claim 1, wherein the at least one opening comprises at least one opening in a side of the tube.

10. The system of claim 9, wherein an end of the portion of the tube inserted into the open cell foam body is closed.

11. The system of claim 1, further comprising at least one clip coupled to the tube and configured to engage a human cheek.

12. An oral feeding apparatus, comprising:
a buccal cavity insert comprising:
an open cell foam body dimensioned for placement in a human mouth between an inside of a cheek and an outside of gums;
a tube inserted into the open cell foam body, wherein a portion of the tube inserted into the open cell foam body has at least one opening; and
wherein the open cell foam body comprises a medial side and a lateral side, the lateral side being shaped to conform to the interior of a cheek.

13. The apparatus of claim 12, wherein the lateral side is shaped to conform to the gums.

14. The apparatus of claim 12, wherein the lateral side is curved inward.

15. The apparatus of claim 12, wherein the medial side is one of: flat and curved outward.

16. The apparatus of claim 12, wherein the at least one opening is at an end of the portion of the tube inserted into the open cell foam body.

17. The apparatus of claim 12, wherein the at least one opening comprises at least one opening in a side of the tube.

18. The apparatus of claim 17, wherein an end of the portion of the tube inserted into the open cell foam body is closed.

19. The apparatus of claim 12, further comprising at least one clip coupled to the tube and configured to engage a human cheek.

20. The apparatus of claim 12, further comprising a backing layer disposed against the medial side.

21. A method for moving fluid to a patient's mouth comprising:
operating a first fluid reservoir on a fluid transfer system to supply fluid to the patient's mouth; the fluid transfer system comprising:
a buccal cavity insert, comprising:
an open cell foam body dimensioned for placement in a human mouth between an inside of a cheek and an outside of gums;
a tube inserted into the open cell foam body, wherein a portion of the tube inserted into the open cell foam body has at least one opening; and
wherein the open cell foam body comprises a medial side and a lateral side, the lateral side being shaped to conform to the interior of a cheek; and
a first fluid reservoir in fluid communication with the tube.

22. The method of claim 21, wherein the fluid transfer system further comprises a second fluid reservoir in fluid communication with the first fluid reservoir; and further comprising the step of:
generating suction at the first fluid reservoir to recharge the fluid in the first fluid reservoir from the second fluid reservoir.

23. The method of claim 21, wherein the first fluid reservoir comprises a squeezable fluid housing, and wherein operating the first fluid reservoir comprises squeezing the fluid housing.

24. The method of claim 21, further comprising:
selecting a first fluid reservoir based on patient parameters.

25. The method of claim 24, wherein patient parameters include an ability to receive fluid, size, facial muscle control, and swallowing ability.

26. The method of claim 21, further comprising:
inserting the fluid distribution pad in the patient's mouth;
positioning the fluid distribution pad in the buccal cavity with the medial side facing the teeth and the lateral side facing the cheek; and
securing the tube to the patient's mouth or cheek with one or more adjustable clips.
